(19) United States
(12) Patent Application Publication
(10) Pub. No.: US 2010/0331746 A1
(43) Pub. Date: Dec. 30, 2010

(54) METHOD OF USING ENOXAPARIN FOR REDUCING THE RISK OF DEATH IN ACUTELY ILL MEDICAL PATIENTS

(75) Inventor: Bruno DESLANDES, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMA S.A., Antony (FR)

(21) Appl. No.: 12/708,676

(22) Filed: Feb. 19, 2010

Related U.S. Application Data
(60) Provisional application No. 61/153,995, filed on Feb. 20, 2009.

(51) Int. Cl. A61K 31/727; A61P 7/02; A61P 9/00; A61H 7/00; A61F 13/08

(52) U.S. Cl. 601/134; 514/56; 602/63

(57) ABSTRACT

The invention relates to a method of using of enoxaparin for reducing the risk of death in acutely ill medical patients.

The invention relates to a method of using enoxaparin for reducing the risk of death in acutely ill medical patients.

Clotting is a defense mechanism preventing excessive loss of blood and ingestion of microbes. Yet, inadvertent formation and dislocation of clots may be harmful: venous thromboembolism (VTE) arises when a thrombus forms in a vein and blocks the blood vessel. This pathology may be asymptomatic (silent) or may ultimately lead to deep vein thrombosis (DVT) or pulmonary embolism (PE), in situations where a thrombus detaches from the vessel, goes in the circulation and blocks in the lower limbs or in the lungs.

Antithrombotic drugs prevent the formation and growth of clots. Heparin and especially Low Molecular Weight Heparins (LMWHs) are the current standard therapy in the management of thromboembolic diseases. Their anticoagulant activity is exerted through inhibition of coagulation factors, mainly activated factor X (FXa) and thrombin (factor IIa).

Enoxaparin obtained marketing approval more than 20 years ago, and is the most widely used LMWH in the world. This drug can safely and effectively prevent and treat venous thromboses, which can occur in the post-operative phase of surgical patients, in cancer patients or in medical patients with restricted mobility, and can treat certain acute arterial thrombotic events, in particular in the case of myocardial infarction.

International VTE prophylaxis guidelines recommend the use of antithrombotic drugs not only for surgical patients, but also for medically ill patients, i.e., patients hospitalized for a medical acute illness. Indeed, the medical patient population is at moderate to high risk of VTE, similar to that of patients undergoing surgery. However there is still a large gap between what is recommended and what is practiced. This gap is even more important when looking at non-western countries with lower health care resources, where the rate of prophylaxis may be negligible. Reasons for this include the underestimation of the risk and the perceived cost of effective prophylaxis, which in turn may be partly explained by a lack of definitive proof of its value, which is based mostly on the reduction of asymptomatic VTE.

But so far, in the case of acutely ill medical patients, no clinical data have shown a direct impact of antithrombotic drugs on mortality reduction.

In the field of unfractionated heparin (UFH), H. Halkin et al. (Ann. Intern. Med., 1982, 96, 561-5) studied its effect on hospital mortality. They showed a statistical difference, but this study was compared to a control arm (no treatment) and was subject to bias as patients were not randomized. B. Gärdlund (Lancet, 1996, 347, 1357-61) did not show any significant effect of heparin versus placebo in infectious disease patients, but the primary endpoint was fatal PE which may require enrollment of a huge number of patients. In addition it is likely that not all the infectious diseases bear the same risk, so these results can not be extrapolated to the whole medical patient population.

R. Dahan et al. (Haemostasis, 1986, 16, 159-64) carried out the first placebo controlled study looking at the effect of LMWH versus placebo. However, it was carried out in an unselected group of elderly patients and was not powered to look at mortality as primary endpoint.

The only study which looked at the effect of LMWH with overall mortality as primary endpoint was conducted by I. Mahé et al. (Eur. J. Clin. Pharmacol., 2005, 61, 347-351). This study, which used a very low dose of nadroparin and enrolled a variety of unselected patients hospitalized in internal medicine wards, failed to show any significant difference versus placebo.

Two other studies were carried out in acutely ill medical patients, but mortality was only a secondary endpoint.

The MEDENOX study (M. M. Samama et al., N. Engl. J. Med., 1999, 341, 793-800) randomized acutely ill medical patients to enoxaparin or placebo and showed a clear reduction of the primary endpoint (VTE events) in favor of enoxaparin. However, no conclusions could be drawn as regards survival rates as the study was not powered adequately.

The PREVENT study (A. Leizorovicz et al., Circulation, 2004, 110, 874-879) was an international, multicenter, randomized, double-blind, placebo-controlled trial which was conducted in 3706 acutely ill medical patients. Patients received either subcutaneous dalteparin or placebo for 14 days and were followed up for 90 days. The primary endpoint was a composite of symptomatic DVT and PE, asymptomatic proximal DVT detected by compression ultrasound and sudden death. A clear reduction of the primary endpoint was shown but there was no difference in mortality as again the study was not designed to show a mortality difference: 2.3% at day 21 and 6% in each group at day 90 (dalteparin/placebo). Most deaths were due to underlying medical conditions, and the causes of death were similar in both groups.

A recent meta-analysis (F. Dentali et al., Annals of Internal Medicine, 2007, 146, 278-288) has concluded that anticoagulant prophylaxis is effective in preventing symptomatic VTE as well as fatal PE but did not show any effect on mortality from all causes. It may be explained by the large number of deaths due to causes that were not related to VTE compared with the small number of deaths attributed to PE. It could also be explained by some limitations that may affect the validity of the studies such as the open design of some studies, which may lead to a higher detection of symptomatic VTE in untreated patients, the usage of different compounds which may have different effects, the inadequate dosage used in some studies, and the different populations selected that may not benefit the same way from the antithrombotic prophylaxis.

It therefore appears that in view of the currently available studies, neither LMWHs nor UFH can be considered as having been shown to reduce mortality in medical patients.

In accordance with the present invention, it has now found that a specific LMWH, namely enoxaparin, reduces mortality in a certain category of patients in need of VTE prophylaxis.

The subject-matter of the invention is a method of using enoxaparin for reducing the risk of death in acutely ill medical patients.

Indeed, it has now been found that administration of enoxaparin to a patient in need thereof reduces the risk of mortality: statistically, the risk of death of the patient is decreased for a patient having been treated with enoxaparin, compared to the risk of death for a patient not having taken such a treatment (treatment being understood as a prophylactic treatment).

The invention therefore relates to a method of using enoxaparin, especially enoxaparin sodium, for reducing the risk of mortality in acutely ill medical patients.

As used herein, “enoxaparin sodium” refers to the low molecular weight heparin (LMWH) approved by the U.S. Food and Drug Administration (FDA), or any other regulatory agency outside of the United States, as Lovenox® (enoxaparin sodium injection), Clexane® or Klexane®, and any LMWH approved by the FDA, or any other regulatory agency outside of the United States, pursuant to an application citing Lovenox® (enoxaparin sodium injection), Clexane® or Klexane® as the listed drug. Enoxaparin sodium is available from sanofi-aventis and sold in the United States in the form of enoxaparin sodium injection under the trademark Lovenox® (Clexane® or Klexane® in some other countries). In general, enoxaparin sodium is obtained by alkaline degradation of heparin benzyl ester derived from porcine intestinal mucosa. Its structure is characterized, for example, by a 2-O-sulfo-4-enepyranosuronic acid group at the non-reducing end and a 2-N,6-O-disulfo-D-glucosamine at the reducing end of the chain. The average molecular weight is about 4500 daltons. The molecular weight distribution is:

| | |
|---|---|
| <2000 daltons | ≦20% |
| 2000 to 8000 daltons | ≧68% |
| >8000 daltons | ≦18% |

Enoxaparin sodium injection is a sterile aqueous solution containing enoxaparin sodium. Enoxaparin sodium injection is available from sanofi-aventis at 100 mg/ml in prefilled syringes (30 mg/0.3 mL pre-filled syringes, 40 mg/0.4 mL pre-filled syringes, 60 mg/0.6 mL pre-filled syringes, 80 mg/0.8 mL pre-filled syringes, and 100 mg/1.0 mL pre-filled syringes), graduated prefilled syringes, multiple-dose vials (300 mg/3.0 mL multi-dose vials), and ampoules (30 mg/0.3 mL). Enoxaparin sodium injection 100 mg/mL concentration contains 10 mg enoxaparin sodium (approximate anti-Factor Xa activity of 1000 IU [with reference to the W.H.O. First International LMWH Reference Standard]) per 0.1 mL water for injection. Enoxaparin sodium injection is also available from sanofi-aventis at 150 mg/ml in graduated prefilled syringes (90 mg/0.6 mL pre-filled syringes, 120 mg/0.8 mL pre-filled syringes, and 150 mg/1.0 mL pre-filled syringes). Enoxaparin sodium injection 150 mg/mL concentration contains 15 mg enoxaparin sodium (approximate anti-Factor Xa activity of 1500 IU [with reference to the W.H.O. First International LMWH Reference Standard]) per 0.1 mL water for injection. The enoxaparin sodium injection prefilled syringes and graduated prefilled syringes are preservative-free and intended for use only as a single-dose injection. There are also multiple-dose vials and those contain 15 mg/1.0 mL benzyl alcohol as a preservative. The pH of the injection is 5.5 to 7.5.

According to the instant invention, “acutely ill medical patients” designates patients hospitalized for an acute illness, excluding patients hospitalized for surgery.

As used herein, “mortality” and “death” refer to all causes of death: either the primary diagnosis for which the patient was hospitalized, pulmonary embolism, hemorrhage, intercurrent illness or any other cause.

In an embodiment, the invention relates to a method of using enoxaparin for reducing the risk of death in acutely ill medical patients, wherein said patients presented one of the following acute medical illnesses which motivated their hospitalization (i.e. prior to receiving a treatment with enoxaparin):
acute decompensation of heart failure,
severe systemic infection and at least one of the following pathologies or physical criteria:
   chronic pulmonary diseases, such as COPD (chronic obstructive pulmonary disease), pulmonary fibrosis or pulmonary restrictive syndrome,
   obesity (being defined with body mass index (BMI)≧30 kg/m²),
   history of VTE, or
   age≧60 years, or
active cancer (defined as being histologically confirmed with an initial diagnosis or recurrence or metastasis within the past 6 months), excluding planned hospitalization for chemotherapy.

In another embodiment, the invention relates to a method of using enoxaparin for reducing the risk of death in acutely ill medical patients, wherein said patients met all of the following criteria prior to their hospitalization (i.e. prior to receiving a treatment with enoxaparin):

1. Male or female≧40 years-old;

2. Hospitalization for at least one of the following medical acute medical illnesses:
acute decompensation of heart failure,
severe systemic infection and at least one of the following:
   chronic pulmonary diseases, such as COPD, pulmonary fibrosis or pulmonary restrictive syndrome,
   obesity (being defined with BMI≧30 kg/m²),
   history of VTE, or
   age≧60 years,
active cancer (defined as being histologically confirmed with an initial diagnosis or recurrence or metastasis within the past 6 months), excluding planned hospitalization for chemotherapy;

3. Anticipated duration of hospitalization for at least 6 days;

4. Health status:
ASA (American Society of Anesthesiologists) Health status score≦3,
ECOG (Eastern Cooperative Oncology Group, as defined in Am. J. Clin. Oncol., 1982, 5, 649-655)≦2 in cancer patient; and

5. Anticipated life expectancy>or equal to 1 week.

In another embodiment, the risk of death is reduced in acutely ill medical patients having taken, during their hospitalization for one of the above illnesses, a treatment with enoxaparin for 6 to 14 days. Said treatment advantageously consists in daily, subcutaneous injections of a 40 mg dose of enoxaparin.

As used therein, “daily” means, for example, administration every 24 hours plus or minus 4 hours.

In another embodiment, said acutely ill medical patients undergo a means of mechanical thromboprophylaxis (for example they may wear Graduated Elastic Stockings) during the treatment period with enoxaparin.

In another embodiment, the risk of death is reduced in acutely ill medical patients at day 30 after start of the treatment with enoxaparin.

In another embodiment, the risk of death is reduced in acutely ill medical patients at day 90 after start of the treatment with enoxaparin.

As used herein, “start of the treatment with enoxaparin” means the day the patient is hospitalized or 1 or 2 days after the patient is hospitalized.

In another embodiment of the instant invention, the risk of death is reduced in an acutely ill medical patient at about day 30, or at about day 90, after hospitalization of said patient.

The invention also relates to a medicament comprising enoxaparin which is useful for reducing the risk of death in acutely ill medical patients.

The invention also relates to a method for reducing the risk of death in acutely ill medical patients, which method comprises administering to such patient a therapeutically effective amount of enoxaparin.

It will be readily apparent to one of ordinary skill in the relevant arts that other suitable modifications and adaptations to the use of enoxaparin described herein are suitable and may be made without departing from the scope of the invention or any embodiment thereof.

Having now described the present invention in detail, the same will be more clearly understood by reference to the following example of the invention, which is included herewith for purposes of illustration only and is not intended to be limiting of the invention.

The following abbreviations shall be used:

BMI: body mass index

BP: blood pressure,

COPD: chronic obstructive pulmonary disease

DVT: deep venous thrombosis

GES: graduated elastic stockings

HAT: heparin associated thrombocytopenia

HIT: heparin induced thrombocytopenia

HITTS: heparin induced thrombotic thrombocytopenia syndrome

LMWH: low molecular weight heparin

PE: pulmonary embolism

sc: subcutaneous

UFH: unfractionated heparin

VTE: venous thromboembolism

The LIFENOX study: International, multi-center, randomized, double blind study to compare the overall mortality in acutely ill medical patients treated with enoxaparin versus placebo in addition to Graduated Elastic Stockings.

1) Study objectives

In this study enoxaparin is compared to placebo in association with Graduated Elastic Stockings (GES). To date enoxaparin is indicated in the prevention of thromboembolic disease. As developing symptomatic VTE may cause a significant burden with long term consequence from the post thrombotic syndrome, it is proposed to use GES in the control arm which have been shown to reduce the risk of DVT, but with no effect on fatal PE. GES shall therefore not affect the primary outcome.

The primary objective is to demonstrate, in patients hospitalized for acute medical illness, that enoxaparin 40 mg sc once daily for 10±4 days with GES is superior to enoxaparin-placebo with GES on overall mortality (all causes) at day 30 after randomization.

The main secondary objective is to compare, in patients hospitalized for acute medical illness, enoxaparin 40 mg sc once daily for 10±4 days with GES versus enoxaparin-placebo with GES on overall mortality at day 90 after randomization.

The other secondary objective is to evaluate the safety of enoxaparin VTE prophylaxis in patients hospitalized for acute medical illness with respect to major hemorrhage, total bleedings, heparin induced thrombocytopenia, adverse events and serious adverse events.

2) Study design

This study is an international, prospective, multicenter, randomized, parallel-groups study of patients hospitalized for acute medical illness fulfilling the inclusion/exclusion criteria. The patients are randomized in 2 groups:
Group A: enoxaparin 40 mg once daily for 6 to 14 days,
Group B: placebo of enoxaparin 40 mg once daily for 6 to 14 days.

The study medication is administered subcutaneously once daily and every 24±4 hours for a minimum of 6 days and up to a maximum of 14 days during the hospitalization for acute medical illness. GES are provided in both groups to be worn at least during the study medication period.

The patient is randomized at baseline visit and followed during the hospitalization up to the discharge. A follow-up visit takes place at day 30 after baseline visit and a second follow-up visit at day 90 after baseline. The total duration of the study for a patient is day 90.

3) Patients

Patients meeting all of the following criteria are suitable for enrollment in the study:

1. Male or female≧40 years-old;

2. Hospitalization within 24 hours prior inclusion for at least one of the following medical acute medical illnesses:
acute decompensation of heart failure,
severe systemic infection and at least one of the following:
   chronic pulmonary diseases (COPD, pulmonary fibrosis, pulmonary restrictive syndrome, . . . ),
   obesity (BMI≧30 kg/m²),
   history of VTE,
   age≧60 years,
active cancer (defined as being histologically confirmed with an initial diagnosis or recurrence or metastasis within the past 6 months) excluding planned hospitalization for chemotherapy;

3. Anticipated duration of hospitalization for at least 6 days; 4. Health status:
ASA (American Society of Anesthesiologists) Health status score≦3,
ECOG (Eastern Cooperative Oncology Group, as defined in Am. J. Clin. Oncol., 1982, 5, 649-655)≦2 in cancer patient;

5. Anticipated life expectancy>or equal to 1 week;

6. Signed written informed consent.

Patients meeting one of the following criteria are excluded for enrollment into the study:

1. Major surgery or major trauma within the previous 6 weeks (orthopedic or trauma surgery to the lower extremities, gastro-intestinal tract, urological, chest, gynecological surgery, . . . );

2. Need for ventilatory support (with intubation required);

3. Symptomatic VTE at enrollment;

4. Multi organ failure;

5. Any evidence of an active bleeding disorder;

6. Contraindication to anticoagulation:
Coagulopathy (acquired or inherited),
Neurosurgery within the past day 30,
Prior history of cerebral hemorrhage at any time,
Known bacterial endocarditis,
Uncontrolled arterial hypertension (systolic BP>200 mm Hg or diastolic BP>110 mm Hg) at 2 successive readings,
Haemostatic abnormalities: baseline platelet count<50,000/mm³, activated partial thromboplastin time (aPTT) 1.5× the upper limit of normal, or International Normalized Ratio (INR)>1.5,
Indication for thrombolytic therapy,
Need for a curative treatment of anticoagulant therapy (LMWH, UFH, oral anticoagulant therapy),
Receiving LMWH or UFH at prophylactic doses for more than 72 hours prior to inclusion (patients receiving LMWH or UFH at prophylactic doses for 72 hours or less prior to entry may be included),
Treatment with oral anticoagulant therapy within 72 hours prior to inclusion;

7. Cerebrovascular accident at inclusion;

8. Patients with prosthetic heart valves;

9. Patients with confirmed cerebral metastases;

10. Known hypersensitivity to heparin or LMWH, or pork-derived products;

11. History of documented episode of heparin or LMWH induced thrombocytopenia and/or thrombosis (HIT, HAT or HITTS);

12. Participation in another clinical trial within the previous 30 days (patients with cancer included in a cancer treatment protocol are authorized to participate only if it is in accordance with local regulation and if they are in follow-up period, with no cancer investigational treatment planned during Lifenox treatment/hospitalization period);

13. Persistent renal failure defined as a documented value of calculated creatinine clearance<30 mL/min on at least 2 occasions 3 days prior to entry into the study;

14. Known or suspected severe anemia of unexplained cause considered clinically relevant by investigator;

15. Spinal or epidural analgesia or lumbar puncture within the preceding 24 hours;

16. Patient unlikely to be compliant (e.g. alcohol, other drug abuse, etc);

17. Woman of childbearing potential not protected by effective contraceptive method of birth control and/or who are unwilling to be tested for pregnancy (pregnancy status should be checked by serum or urine pregnancy testing prior to exposure to the investigational product);

18. Refusal or inability to give informed consent to participate in the study;

19. Inability to be followed-up after discharge until day 90 after randomization;

4) Treatments

Sanofi-aventis supplies and manufactures the blinded treatments for this study, i.e. enoxaparin sodium 40 mg/0.4 mL (Lovenox®/Clexane®) and matching placebo. The treatments are provided in pre-filled, single-dose syringes for subcutaneous injection. Lovenox®/Clexane® 40 mg injection is a sterile solution containing enoxaparin sodium. Lovenox®/Clexane® contains 10 mg enoxaparin sodium per 0.1 mL Water for Injection. For the placebo, each syringe contains normal (0.9%) saline, identical in appearance to the above.

According to their randomized assignments, patients receive either enoxaparin 40 mg or matching placebo once daily and every 24±4 hours during a minimum of 6 days in hospital ward. The duration of treatment lasts between 6 days and up to a maximum of 14 days (10±4 days).

Mechanical thromboprophylaxis with GES is used in the 2 groups during the study medication period. They should be worn continuously until at least the end of study treatment period (6 to 14 days).

What is claimed is:

1. A method for reducing the risk of death in an acutely ill medical patient, the method comprising administering an effective amount of enoxaparin to said patient.

2. The method according to claim 1, wherein said patient is hospitalized for at least one of the following acute medical illnesses:
acute decompensation of heart failure,
severe systemic infection and at least one of the following: chronic pulmonary disease, obesity, history of venous thromboembolism, or age equal to or above 60 years, or
active cancer, excluding planned hospitalization for chemotherapy.

3. The method according to claim 1, wherein said patient met all of the following criteria:
Male or female of age equal to or above 40 years-old;
Hospitalization for at least one of the following acute medical illnesses:
acute decompensation of heart failure,
severe systemic infection and at least one of the following: chronic pulmonary disease, obesity, history of venous thromboembolism, or age equal to or above 60 years,
active cancer, excluding planned hospitalization for chemotherapy;
Anticipated duration of hospitalization for at least 6 days;
Health status:
ASA Health status score≦3 (American Society of Anesthesiologists),
ECOG≦2 in cancer patient (Eastern Cooperative Oncology Group); and
Anticipated life expectancy greater or equal to 1 week.

4. The method according to claim 1, wherein said patient is treated with enoxaparin for 6 to 14 days.

5. The method according to claim 4, wherein the daily dose of enoxaparin is 40 mg.

6. The method according to claim 1, wherein said patient undergoes mechanical thromboprophylaxis during the treatment period with enoxaparin.

7. The method according to claim 6, wherein said thromboprophylaxis comprises the use of graduated elastic stockings.

8. The method according to claim 1, wherein the risk of death is reduced at day 30 after the start of the treatment with enoxaparin.

9. The method according to claim 1, wherein the risk of death is reduced at day 90 after the start of the treatment with enoxaparin.
